(19) United States
(12) Patent Application Publication
(10) Pub. No.: US 2025/0388893 A1
(43) Pub. Date: Dec. 25, 2025

(54) DUAL EXPRESSION VECTOR AND METHOD

(71) Applicant: Huazhong Agricultural University, Wuhan (CN)

(72) Inventors: Lin LI, Wuhan (CN); Xiaoyang SHANG, Wuhan (CN)

(73) Assignee: Huazhong Agricultural University, Wuhan (CN)

(21) Appl. No.: 19/000,380

(22) Filed: Dec. 23, 2024

Related U.S. Application Data
(63) Continuation of application No. PCT/CN2024/121281, filed on Sep. 26, 2024.

(30) Foreign Application Priority Data
Jun. 20, 2024 (CN) 2024108035820

(51) Int. Cl. C12N 15/10; C12N 15/81

(52) U.S. Cl. CPC C12N 15/1055; C12N 15/1082; C12N 15/81; C12N 2800/102

(57) ABSTRACT

A dual expression vector and a method are provided. The dual expression vector has a first multiple cloning site and a second multiple cloning site. The genes of the different proteins could be inserted into the first multiple cloning site and the second multiple cloning site of the dual expression vector for testing the interaction between the different proteins.

### CROSS-REFERENCE TO RELATED APPLICATIONS

This disclosure claims priority to Chinese Patent Application NO: 2024108035820, filed with China Intellectual Property Office on Jun. 20, 2024, the entire contents of which are incorporated herein by reference.

### SEQUENCE LISTING

The sequence listing xml file submitted herewith, named “Sequence_Listing.xml”, created on Dec. 11, 2024, and having a file size of 68,240 bytes, is incorporated by reference herein.

### TECHNICAL FIELD

This application relates to the protein interaction in yeast, specifically involving a dual expression vector and method.

### BACKGROUND

Protein to protein interaction (PPI) forms a major component of the biochemical reaction network within cells. Yeast two-hybrid system is widely used in the study of PPI. When the bait protein binds to the gene promoter of the prey protein and initiates the expression of the gene of the prey protein in yeast cells, if the gene expression of the prey protein is detected, it indicates that there is interaction between the prey protein and the bait protein; otherwise, there is no interaction between the two proteins. The micro-quantified and arrayed yeast two-hybrid system can be used to study protein-protein interactions on a large scale. At present, the expression vector for yeast two-hybridization test is mainly constructed by enzyme-ligation and Gateway cloning techniques.

However, when the protein interaction is identified by the yeast two-hybrid system, at least two vectors, such as a capture vector and a bait vector, need to be constructed on the foreign genes under study, and the two vectors are transferred into two yeast strains respectively for expression and hybridization culture, so as to determine whether the two are hybridized. When it is necessary to test multiple pairs of proteins, it is necessary to construct multiple vectors accordingly. The traditional yeast two-hybrid system will greatly increase the workload of identification of multiple protein interactions or protein library interactions, and the efficiency of identification is severely limited.

### SUMMARY

One aspect, embodiments provide a dual expression vector. This dual expression vector has a first sequence and a second sequence. The first sequence includes a orderly linked sequence consisting of a first multiple cloning site, a coding region of the GALA activation domain, a first promoter, a first operon, and a first initiator. The first promoter, the coding region of the GAL4 activation domain, and the first multiple cloning site have the same transcribed direction. The first promoter promotes the expression of the coding region of the GAL4 activation domain and the first target gene to be inserted at the first multiple cloning site. The second sequence includes a orderly linked sequence consisting of a second multiple cloning site, a coding region of the GAL4 binding domain, a second promoter, a second operon, and a second initiator. The second promoter, the coding region of the GAL4 binding domain, and the second multiple cloning site have the same transcribed direction. The second promoter promotes the expression of the coding region of the GAL4 binding domain and the second target gene to be inserted at the second multiple cloning site. The first sequence and the second sequence are joined to form a circular DNA molecule.

One aspect, embodiments provide a method for preparing a dual expression vector. The method includes: providing a basic vector having a first multiple cloning site and a second multiple cloning site; synthesizing a terminator; and inserting the terminator between the transcription end of the first multiple cloning site and the transcription end of the second multiple cloning site.

The term “dual expression vector” refers to a vector capable of expressing two genes simultaneously in yeast cells. The vector is used to test the interaction between these two genes in yeast.

### BRIEF DESCRIPTION OF DRAWINGS

FIG. 1 shows the schematic diagram of a dual expression vector according to embodiments.

FIG. 2 shows the schematic diagram of a dual expression vector according to embodiments.

FIG. 3 shows the plate images on SD/-Trp/-Leu and SD/-Trp/-Leu/-Ade/-His of the yeast transformants diluted in 1, 10, and 100 times respectively. The positive control group uses the dual expression vector carrying both P53 and T genes (BD-P53 AD-T) and shows the state of interaction. The dual expression vector carrying only P53 gene or only T gene (AD-T) do not show the state of interaction, serving as negative controls. The empty dual expression vector also did not show the state of interaction.

FIG. 4 shows the plate images on SD/-Trp/-Leu and SD/-Trp/-Leu/-Ade/-His of the yeast transformants diluted in 1, 10, and 100 times, respectively. The interaction between the BIF1 gene and the BIF4 gene, the interaction between the KRN2 gene and the DUF1644 gene, the interaction between the GIF1 gene and the GRF1 gene, the interaction between the MSCA1 gene and the FEA4 gene, and the interaction between the KNR6 gene and the AGAP gene are shown in FIG. 4.

FIG. 5 shows the plate images on SD/-Trp/-Leu and SD/-Trp/-Leu/-Ade/-His of a test example of yeast transformants carrying BD library and AD library, diluted in 10, 100, 1000 times, respectively.

### DETAILED DESCRIPTION OF THE EMBODIMENTS

In order to make the purpose, technical scheme and advantAGAPes of this application more clear, the following combined embodiments are further explained in detail. It will be understood that the specific embodiments described herein are intended only to explain the application and are not intended to qualify the application. The reAGAPents not separately described in detail in this application are all routine reAGAPents and can be obtained from commercial sources. The methods not specified in detail are routine experimental methods and can be known from the prior art.

### Dual Expression Vector

Some embodiments provide a dual expression vector. This dual expression vector has a first sequence and a second sequence. The first sequence includes a orderly linked sequence consisting of a first multiple cloning site, a coding region of the GAL4 activation domain, a first promoter, a first operon, and a first initiator. The first promoter, the coding region of the GAL4 activation domain, and the first multiple cloning site have the same transcribed direction. The first promoter promotes the expression of the coding region of the GALA activation domain and the first target gene to be inserted at the first multiple cloning site. The second sequence includes a orderly linked sequence consisting of a second multiple cloning site, a coding region of the GAL4 binding domain, a second promoter, a second operon, and a second initiator. The second promoter, the coding region of the GAL4 binding domain, and the second multiple cloning site have the same transcribed direction. The second promoter promotes the expression of the coding region of the GAL4 binding domain and the second target gene to be inserted at the second multiple cloning site. The first sequence and the second sequence are joined to form a circular DNA molecule.

In some embodiments, the dual expression vector also has a terminator. The terminator is located between the transcription terminal of the first multiple cloning site and the transcription terminal of the second multiple cloning site. In some embodiments, the terminator is SEQ ID NO:11. The terminator terminates the expression of the coding region of the GALA activation domain in the first sequence and the expression of the first target gene to be inserted in the first multiple cloning site, and the expression of the GAL4 binding domain and the second target gene inserts in the second multiple cloning site, respectively.

In some embodiments, the first multiple cloning site has at least two restriction enzyme sites. The second multiple cloning site also has at least two restriction enzyme sites too.

In some embodiments, the dual expression vector also has one T7 promoter inserted between the first multiple cloning site and the coding region of the GAL4 activation domain, and another T7 promoter between the second multiple cloning site and the coding region of the GAL4 binding domain, respectively.

In some embodiments, both the first promoter and the second promoter are promoters suitable for yeast.

In some embodiments, the first promoter and the second promoter are independently selected from GK (phosphoglycerol kinase) promoter, GAP (glyceraldehyde 3-phosphate dehydrogenase) promoter, ADH (alcohol dehydrogenase) promoter, G3P (glyceraldehyde 3-phosphate dehydrogenase) promoter, ICL1 (isocitrate lyase) promoter, AOX1 (alcohol oxidase 1) promoter, TEF (transcription elongation factor EF-1a) type promoters, GALI (galactokinase) type promoters, GALI (galactokinase gene) type promoters, Trp1 (tryptophan operon) promoters or promoters derived from them.

In some embodiments, the first promoter is an ADH1 promoter.

In some embodiments, the second promoter is a truncated ADH1 promoter.

In some embodiments, the first operon includes a first operator gene and a promoter for promoting the first operator gene. The second operon includes a second operator gene and a promoter for promoting the second operator gene.

In some embodiments, the first operator gene and the second operator gene are independently selected from LEU2, TRP1, or HIS. For example, the first operator gene is LEU gene, and its promoter is the LEU2 promoter. For example, the second operator gene is TRP gene, and its promoter is the TRP1 promoter.

In some embodiments, the second sequence also includes an third initiator located between the second promoter and the second gene operator, and a resistance gene connected to the transcriptional start end of the second initiator. The resistance gene has a transcription direction same to the third initiator. The third initiator has a transcription direction opposite to the GALA binding domain.

In some embodiments, the resistance gene is selected from one of Ampicillin, Tetracycline, Chloramphenicol, Streptomycin, Hygromycin, Spectinomycin, card Kanamycin, Blasticidin, Geneticin, Hygromycin B, Mycophenolic acid Acid, Puromycin, Zeocin, or Neomycin.

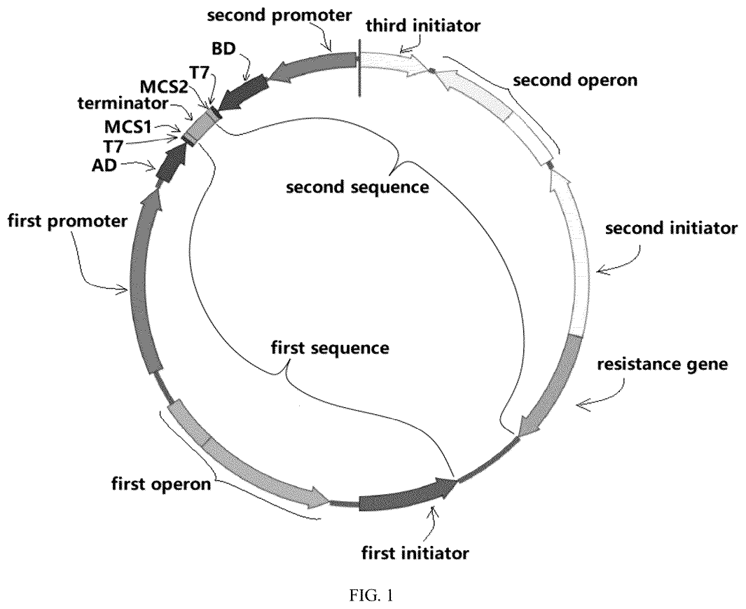

As shown in FIG. 1, the nucleotide sequence of the dual expression vector starts with a third initiator shown as SEQ ID NO. 1. It is sequentially arranged through the second operon shown as SEQ ID NO:2, the second initiator shown as SEQ ID NO:3, the Kanamycin resistance gene shown as SEQ ID NO:4, the first initiator shown as SEQ ID NO:5, the first operon shown as SEQ ID NO:6, the first promoter shown as SEQ ID NO:7, the GAL4 activation domain shown as SEQ ID NO:8, the T7 promoter shown as SEQ ID NO:9, the MCS1 shown as SEQ ID NO:10, the terminator shown as SEQ ID NO:11, the MCS2 shown as SEQ ID NO:12, the GAL4 DNA binding domain shown as SEQ ID NO:13, and the second promoter shown as SEQ ID NO:14. And the nucleotide sequences shown in the sequence listing xml file are all forward strand sequences in this order.

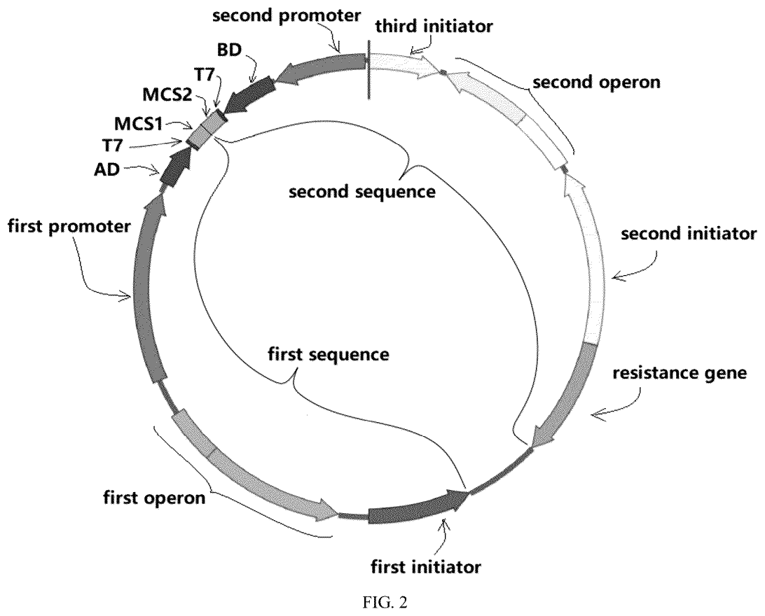

FIG. 2 shows the structure of the dual expression vector according to some embodiments. The first sequence includes a orderly linked sequence consisting of a first multiple cloning sites (MCS1, SEQ ID NO: 10), a T7 promoter (T7, SEQ ID NO: 9), a coding region of the GAL4 activation domain (AD, SEQ ID NO: 8), a first promoter (SEQ ID NO: 7), a first operon (SEQ ID NO: 6), and a first initiator (SEQ ID NO: 5). The second sequence includes a orderly linked sequence consisting of a second multiple cloning sites (MCS2, SEQ ID NO: 12), a T7 promoter (T7, SEQ ID NO: 9), a coding region of the GAL4 DNA binding domain (BD, SEQ ID NO: 13), a second promoter (SEQ ID NO: 14), a third initiator (SEQ ID NO: 1), a second operon (SEQ ID NO: 2), a second initiator (SEQ ID NO: 3), and a kanamycin resistance gene (Kana, SEQ ID NO: 4).

As shown in FIG. 1 and FIG. 2, the transcription direction of each region on the dual expression vector is indicated by its arrow.

As shown in FIG. 1 and FIG. 2, the dual expression vector also has a sequence as SEQ ID NO:15 between the kanamycin resistance gene and the first initiator, and a sequence as SEQ ID NO:16 between the first operon and the first promoter.

### Prepare the Dual Expression Vector

Some embodiments provide a method for preparing the dual expression vector according to the above embodiments. The method includes: providing a basic vector consisting of the first sequence and the second sequence; synthesizing the terminator; and inserting the terminator between the transcription end of the first sequence and the transcription end of the second sequence to form a circular DNA molecule.

Other embodiments also provide a method for preparing the dual expression vector according to the above embodiments. The method includes: synthesizing a third fragment orderly including the first multiple cloning site, the T7 promoter, the coding region of the GAL4 activation domain and the first promoter; synthesizing a fourth fragment including the first operon; synthesizing a fifth fragment including the first initiator, the resistance gene and the second initiator; synthesizing a sixth fragment including the second initiator, the second operon, the third initiator, the second promoter, the coding region of the GAL4 binding domain, the T7 promoter and the second multiple cloning site; homologously recombining the third fragment, the fourth fragment, the fifth fragment and the sixth fragment; transferring the homologous recombination product into Escherichia coli; screening positive colonies from the transformants; extracting the basic vector from the culture of the positive colonies.

In some embodiments, the step of synthesizing the third fragment includes: amplifying a pGADT7 plasmid (Coolaber) by primers F3 (SEQ ID NO:17) and R3 (SEQ ID NO:18), and a high-fidelity enzyme; subjecting the amplification product to the gel electrophoresis; and recovering the third fragment (SEQ ID NO:19) from the gel electrophoresis.

In some embodiments, the step of synthesizing the fourth fragment includes: amplifying a pGADT7 plasmid by primers F4 (SEQ ID NO:20) and R4 (SEQ ID NO:21), and a high-fidelity enzyme; gel electrophoresis the amplification product; and recovering the fourth fragment (SEQ ID NO:22) from the gel electrophoresis.

In some embodiments, the step of synthesizing the fifth fragment includes: amplifying a pGBKT7 plasmid (Coolaber) by primers F5 (SEQ ID NO:23) and R5 (SEQ ID NO:24), and a high-fidelity enzyme; gel electrophoresis the amplification product; and recovering the fifth fragment (SEQ ID NO:25) from the gel electrophoresis.

In some embodiments, the step of synthesizing the sixth fragment includes: amplifying the pGBKT7 plasmid by primers F6 (SEQ ID NO:26) and R6 (SEQ ID NO:27), and a high-fidelity enzyme; gel electrophoresis the amplification product; and recovering the sixth fragment (SEQ ID NO:28) from the gel electrophoresis.

In these embodiments, PCR amplification is performed by using the 2×Phanta Max Master Mix High Fidelity Enzyme and ReAGAPent Kit from Vazyme, and the instructions for use can be found on the company's website at <https://www.vazyme.com/product/120.html>. The PCR products could be recovered and purified by using the FastPure Gel DNA Extraction Mini Kit from Vazyme, and the instructions for use could be found on the company's website at <https://www.vazyme.com/companyfile/2149.html>.

The steps also include: mixing 40 ng of the third fragment, 36 ng of the fourth fragment, 70 ng of the fifth fragment, 66 ng of the sixth fragment, and 10 μL of 2×CE Mix (from Vazyme ClonExpress ultra One Step Cloning Kit V2) in a total volume of 20 μL; performing the mixture at 50° C. for 30 minutes; cooling down to 4° C. or immediately placing on ice for cooling; transforming the recombinant into Escherichia coli to obtain positive clones; cultivating the positive colonies; extracting the basic vector from the culture of the positive colonies.

### Test the Protein-Protein Interactions

Some embodiments provide a method for testing the protein-protein interactions. The methods include: separately amplifying the target genes of the two different proteins; inserting the target genes of the two different proteins into the first and second multi-cloning sites of the dual expression vector, respectively, to obtain a recombinant vector carrying the target genes of the two different proteins; transforming the recombinant vector into yeast to obtain a recombinant yeast; and testing the interaction between the two proteins on the growth state of the recombinant yeast on a selective culture medium.

In some embodiments, the yeast is Y2H yeast.

In some embodiments, the selective medium is selected from SD/-Trp/-Leu (product number: PM2220, Coolaber Company), SD/-His/-Leu/-Trp (product number: PM2112, Coolaber Company), SD/-Trp/-Leu/-Ade/-His (product number: PM2112, Coolaber Company), and SD/-Trp/-Leu/-Ade/-His+3-AT. The product number of aureobasidin A (3-AT) is CA2332G. SD/-Trp/-Leu/-Ade/-His+3-AT is adding 10-20 mM 3A-T to the SD/-Trp/-Leu/-Ade/-His medium. In some embodiments, SD/-Trp/-Leu/-Ade/-His+3-AT is adding 20 mM 3A-T to the SD/-Trp/-Leu/-Ade/-His medium.

In some embodiments, the construction process of the recombinant vector includes:

### 1. Linearize the Dual Expression Vector

In some steps, a linearized dual expression vector according to the above embodiments were prepared by digesting with enzymes BspDI and NdeI. The digestion reaction mixture contains 5 μg of the dual expression vector, 5 μL of BspDI (NEB), 5 μL of NdeI (NEB), 10 μL of 10×cutsmart buffer and 85 μL of deionized water in a total of 100 μL. The mixture was incubated at 30° C. for 4 hours, and tested by a gel electrophoresis. And the linearized dual expression vector could be recovered from the gel electrophoresis with the Cycle Pure Kit purification kit (Omega Bio-tek).

### 2. Prepare the First Target Gene and Second Target Gene

The first target gene and second target gene could be prepared by PCRs with primers listed in Table 1, gel electrophoresed the PCR products, and recovered from the gel electrophoresis with the FastPure Gel DNA Extraction Mini Kit (NovAGAPen). The PCR reaction mixture could contain 1.0 μL template, 22 μL deionized water, 1.0 μL upstream primer, 1.0 μL downstream primer, 25 μL and 2×Phanta Max Master Mix in a total of 50 μL. The PCR reaction steps could include: 1 cycle (95° C., 3 minutes), 34 cycles (98° C., 20 seconds; 56° C., 2 minutes; 72° C., 3 minutes), 1 cycle (72° C., 5 minutes) and 1 cycle (4° C., 1 minute).

TABLE 1
Primers of First Target Gene and Second Target Gene

| Target gene and | Primer | |
|---|---|---|
| number | Name | Sequence (5′→3′) |
| P53,Gene ID: 7157, | P53-F | tgatctcagaggaggacctgctgcatatgcctgtcaccga, SEQ ID NO: 29 |
| Sequence | P53-R | cgtatgccgtcttctgcttgcagcaggggatgcagaggca, SEQ ID NO: 30 |
| ID: NM_000546.6 | | |
| T(Acronym T), Gene | T-F | acgacgtaccagattacgctagegccgccatggagtaccc, SEQ ID NO: 31 |
| ID: 29031019, | T-R | tcggtggtcgccgtatcatttgtttcaggttcagggggag, SEQ ID NO: 32 |
| Sequence number | | |
| NC_001669.1 | | |
| KNR6, Sequence | KNR6-F | tgatctcagaggaggacctgatgagttgtttttctcacct, SEQ ID NO: 33 |
| ID: GRMZM2G119714 | KNR6-R | cgtatgccgtcttctgcttgctagtegcgctccgatatctegg, SEQ ID NO: 34 |
| AGAP, Sequence | AGAP-F | tcggtggtcgccgtatcatttcactggctgagcggcacaca, SEQ ID NO: 35 |
| ID: GRMZM2G176495 | AGAP-R | acgacgtaccagattacgctatggaacgacaggggatgga, SEQ ID NO: 36 |
| MSCA1, Sequence | MSCA1-F | tgatctcagaggaggacctgatgctgcggatggaggtgca, SEQ ID NO: 37 |
| ID: GRMZM2G442791 | MSCA1-R | cgtatgccgtcttctgcttgctagagccagagcgcgccggcct, SEQ ID NO: 38 |
| FEA4, Sequence | FEA4-F | tcggtggtcgccgtatcatttcaatccgteggccgcgtca, SEQ ID NO: 39 |
| ID: Zm00001d037317 | FEA4-R | acgacgtaccagattacgctatgcatcgtcagccatctcc, SEQ ID NO: 40 |
| GIF1, Sequence | GIF1-F | tgatctcagaggaggacctgatgcagcagcaacacctgat, SEQ ID NO: 41 |
| ID: GRMZM2G180246 | GIF1-R | cgtatgccgtcttctgcttgctaactgccttcttcctcggtgc, SEQ ID NO: 42 |
| GRF1, Sequence | GRF1-F | tcggtggtcgccgtatcatttcaccttcggtccgtggccag, SEQ ID NO: 43 |
| ID: GRMZM2G034876 | GRF1-R | acgacgtaccagattacgctatggcgatgccgtatgcctc, SEQ ID NO: 44 |
| KRN2, Sequence | KRN2-F | tgatctcagaggaggacctgatggaagggtgccaactgct, SEQ ID NO: 45 |
| ID: Zm00001eb073740 | KRN2-R | cgtatgccgtcttctgcttgctactggtgcgccggtaagc, SEQ ID NO: 46 |
| DUF1644, Sequence | DUF1644-F | tcggtggtcgccgtatcatt tcatctctcagctgcctcgc, SEQ ID NO: 47 |
| ID: Zm00001eb182660 | DUF1644-R | acgacgtaccagattacgctatggcaagatcaccaaaagg, SEQ ID NO: 48 |
| BIF1, Sequence | BIF1-F | tgatctcagaggaggacctgatgtcggtggatacggagcg, SEQ ID NO: 49 |
| ID: Zm00001d008749 | BIF1-R | cgtatgccgtcttctgcttgtcaaaacagcgacgaagtac, SEQ ID NO: 50 |
| BIF4, Sequence | BIF4-F | tcggtggtcgccgtatcatttcagagttgagactccaggg, SEQ ID NO: 51 |
| ID: Zm00001d037691 | BIF4-R | acgacgtaccagattacgctatggcttggaatggccgctt, SEQ ID NO: 52 |

### 3. Recombine the First Target Gene, the Second Target Gene and the Linearized Dual Expression Vector

The recombinant vector was obtained by inserting the first target gene (Bait) and the second target gene (Pery) into the first multiple cloning site and the second multiple cloning site respectively of the linearized dual expression vector. Among them, the recombination reaction was carried out using the ClonExpress μLtra One Step Cloning Kit V2 kit. The recombination reaction mixture could be shown in Table 2. The recombination reaction mixture was incubated at 50° C. for 30 minutes; and then cooled to 4° C. or immediately placed on ice.

TABLE 2
Recombination reaction mixture

| Ingredient | Amount | |
|---|---|---|
| First target gene | 40 | ng(1 μL) |
| Second target gene | 40 | ng(1 μL) |
| Terminator | 10 | ng(1 μL) |
| Linearized basic vector | 200 | ng(1 μL) |
| Deionized water | 85 | ng(1 μL) |
| 2 × CE Mix | 5 | μL |
| Total | 10 | μL |

### 4. Transform Into Yeast

5-10 μL of the recombinant product according to above steps were uniformly mixed in 100 μL of competent Y2H yeast cells, left standing on ice for 30 minutes, heat shocked in a 42° C. water bath for 30 seconds, and cooled on ice for 2-3 minutes. The obtained mixture was added to 900 μL of SOC or LB liquid medium (without adding antibiotics), and cultured at 37° C. and 200-250 rpm for 1 hour. The obtained culture solution was centrifuged at 5000 rpm for 5 minutes, and the obtained supernatant was spread on a resistant LB plate and cultured upside down at 37° C. for 12-16 hours. The colonies on the plates were subjected to colony PCR detection, and the obtained positive colonies were cultured. The dual expression vector was extracted from the culture of the positive colonies.

### 5. Point-to-Point Test for Exogenous Genes

The dual expression vector was used to test the interaction effect of the known interacting genes P53 and T. A recombinant vector was prepared by inserting the genes P53 and T into the first multiple cloning site and the second multiple cloning site of the dual expression vector according to the above embodiments. And the obtained recombinant vector was transformed into Y2H yeast cells. The obtained transformants were spread on selective deficiency medium SD/-Trp/-Leu and SD/-Trp/-Leu/-Ade/-His as positive controls. Another dual expression vector without the first target gene and the second target gene was transformed into Y2H yeast cells. The obtained transformants were spread on selective deficiency medium SD/-Trp/-Leu and SD/-Trp/-Leu/-Ade/-His as negative controls.

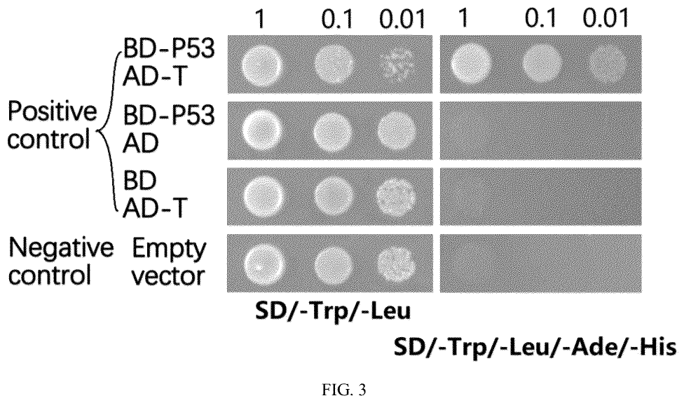

As shown in FIG. 3, the transformants carrying the recombinant vector with only the P53 gene, as well as the transformants carrying the recombinant vector with only the T gene, grow on SD/-Trp/-Leu, but not on SD/-Trp/-Leu/-Ade/-His. On the other hand, the transformants carrying the recombinant vector with both the P53 gene and the T gene grow on SD/-Trp/-Leu and SD/-Trp/-Leu/-Ade/-His. This indicates that the dual expression vector realizes the simultaneous expression of two foreign genes in the same yeast cell, thereby potentially producing interactions. The dual expression vector could be developed as a new tool for protein interaction research with improving research efficiency.

### 6. Other Point-to-Point Test for Exogenous Genes

The dual expression vector provided in the embodiments of this application was used to test the interaction effects of the known interacting genes BIF1 and BIF4 (Galli M, Liu Q, Moss B L, et al. Auxin signaling modules regulate maize inflorescence architecture. Proc Natl Acad Sci USA. 2015; 112(43):13372-13377. doi:10.1073/pnas.1516473112), the interaction effects of the known interacting genes KRN2 and DUF1644 (Chen W, Chen L, Zhang X, et al. Convergent selection of a WD40 protein that enhances grain yield in maize and rice. Science. 2022; 375(6587):eabg7985. doi:10.1126/science.abg7985), the interaction effects of the known interacting genes GIF1 and GRF1 (Zhang D, Sun W, Singh R, et al. GRF-interacting factor1 Regulates Shoot Architecture and Meristem Determinacy in Maize. Plant Cell. 2018; 30(2):360-374. doi:10.1105/tpc.17.00791), the interaction effects of the known interacting genes MSCA1 and FEA4, and the interaction effects of the known interacting genes KNR6 and AGAP (Jia H, Li M, Li W, et al. A serine/threonine protein kinase encoding gene KERNEL NUMBER PER ROW6 regulates maize grain yield. Nat Commun. 2020; 11(1):988. doi:10.1038/s41467-020-14746-7).

The recombinant vector carrying the first target gene being BIF1 and the second target gene being BIF4, the recombinant vector carrying the first target gene being KRN2 and the second target gene being DUF1644, the recombinant vector carrying the first target gene being GIF1 and the second target gene being GRF1, the recombinant vector carrying the first target gene being MSCA1 and the second target gene being FEA4, and the recombinant vector carrying the first target gene being KNR6 and the second target gene being AGAP were constructed respectively. These obtained recombinant vectors were transformed into Y2H yeast cells respectively, and the obtained transformants were spread on selective deficiency medium SD/-Trp/-Leu and selective deficiency medium SD/-Trp/-Leu/-Ade/-His.

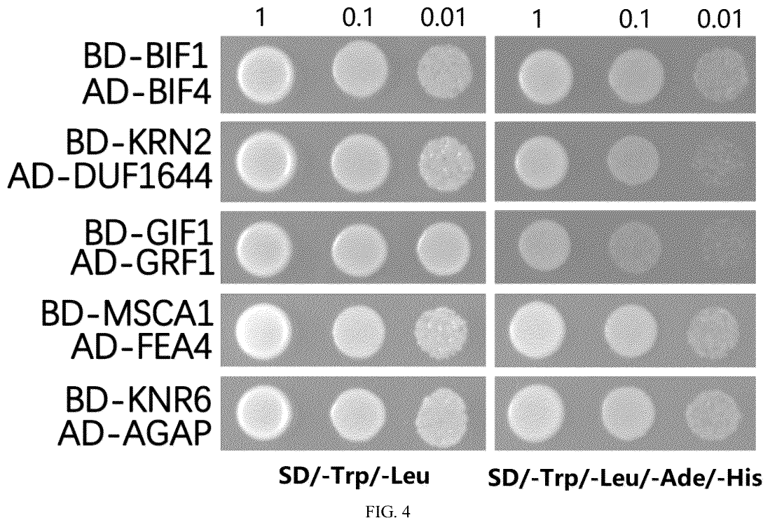

As shown in FIG. 4, the transformants carrying simultaneously these first target gene and second target gene grow on SD/-Trp/-Leu/-Ade/-His. This further demonstrates that the dual expression vector could successfully detect and verify the interaction of genes.

### 7. Test the Interactions Between the Exogenous Gene Libraries

Other recombinant vectors were prepared by inserting the multiple first target genes (such as gene libraries) into the first multiple cloning site and the multiple second target genes (such as gene libraries) into the second multiple cloning site of the dual expression vector according to the above embodiments. These recombinant vectors could be transformed into yeast for testing the interactions between these exogenous gene libraries.

For example, P53, BIF1, KRN2, GIF1, MSCA1 and KNR6 were equally proportionally mixed as the first target genes to form a BD library. T, BIF4, DUF1644, GRF1, FEA4 and AGAP were equally proportionally mixed as the second target genes to form an AD library. The BD library and the AD library were cloned onto the dual expression vector through the multi-fragment recombination reaction system shown in Table 2 to obtain a recombinant vector carrying both the BD library and the AD library. The obtained recombinant vectors were transferred into Y2H yeast cells, and the obtained transformants were spread on selective deficiency medium SD/-Trp/-Leu and selective deficiency medium SD/-Trp/-Leu/-Ade/-His. The obtained yeast cells were used to test the interaction between multiple proteins expressed by the BD library and the AD library.

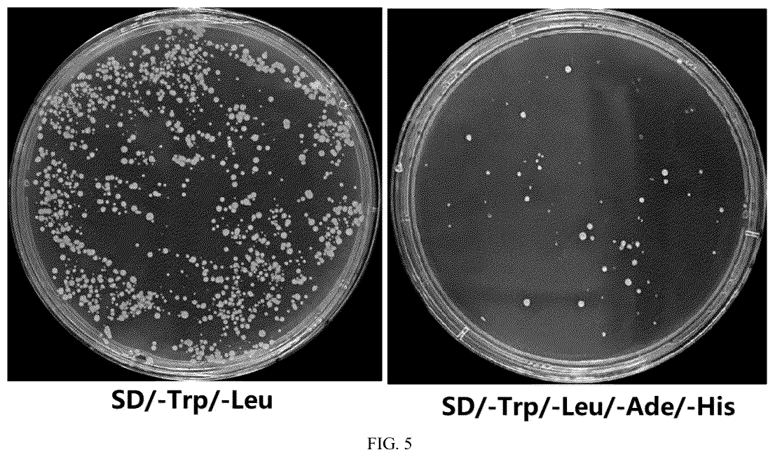

FIG. 5 shows the growth conditions of the transformants containing the recombinant vector on SD/-Trp/-Leu and SD/-Trp/-Leu/-Ade/-His. It could indicate that an interaction occurred between the BD library and the AD library.

All above has said the dual expression vector could not only as a tool to test the interaction of two target genes, but also as a tool to test the interaction of multiple gene libraries. The yeast double expression vector can improve the efficiency of protein interaction test for large-scale libraries.

As mentioned above, these are only the preferred specific implementation methods of this application. However, the protection scope of this application is not limited to this. Any person skilled in the art can easily think of changes or substitutions within the technical scope disclosed in this application, which should be covered within the protection scope of this application.

What is claimed is:

1. A dual expression vector comprising:
a first sequence, said first sequence comprises the orderly linked sequence comprising a first multiple cloning site, a coding region of the GAL4 activation domain, a first promoter, a first operon, and a first initiator, said first promoter, said coding region of the GAL4 activation domain, and said first multiple cloning site have the same transcribed direction, said first promoter promotes the expression of the coding region of the GAL4 activation domain and the target genes to be inserted at said first multiple cloning site;
a second sequence, said second sequence comprises the orderly linked sequence comprising a second multiple cloning site, a coding region of the GAL4 binding domain, a second promoter, a second operon, and a second initiator, said second promoter, said coding region of the GAL4 binding domain, and said second multiple cloning site have the same transcribed direction, said second promoter promotes the expression of the coding region of the GAL4 binding domain and the target genes to be inserted at said second multiple cloning site;
said first sequence and said second sequence are joined to form a circular DNA molecule.

2. The dual expression vector according to claim 1, wherein said dual expression vector further comprises a terminator, said terminator is located between the transcription terminal of said first multiple cloning site and the transcription terminal of said second multiple cloning site.

3. The dual expression vector according to claim 2, wherein said terminator is SEQ ID NO:11.

4. The dual expression vector according to claim 1, wherein said first multiple cloning site comprises at least two restriction enzyme sites.

5. The dual expression vector according to claim 1, wherein said second multiple cloning site comprises at least two restriction enzyme sites.

6. The dual expression vector according to claim 1, wherein said dual expression vector also comprises one T7 promoter between said first multiple cloning site and said coding region of the GAL4 activation domain, and another T7 promoter between said second multiple cloning site and said coding region of the GAL4 binding domain, respectively.

7. The dual expression vector according to claim 1, wherein said coding region of the GAL4 activation domain is SEQ ID NO:8, said coding region of the GAL4 binding domain is SEQ ID NO:13.

8. The dual expression vector according to claim 1, wherein said first promoter is elected from phosphoglycerol kinase promoter, glyceraldehyde 3-phosphate dehydrogenase promoter, alcohol dehydrogenase promoter, glyceraldehyde 3-phosphate dehydrogenase promoter, isocitrate lyase promoter, alcohol oxidase 1 promoter, transcription elongation factor EF-1a type promoters, galactokinase type promoters, galactokinase gene type promoters, or tryptophan operon promoter;
said second promoter is elected from phosphoglycerol kinase promoter, glyceraldehyde 3-phosphate dehydrogenase promoter, alcohol dehydrogenase promoter, glyceraldehyde 3-phosphate dehydrogenase promoter, isocitrate lyase promoter, alcohol oxidase 1 promoter, transcription elongation factor EF-1a type promoters, galactokinase type promoters, galactokinase gene type promoters, or tryptophan operon promoter.

9. The dual expression vector according to claim 1, wherein said first promoter is SEQ ID NO:7, said second promoter is SEQ ID NO:14.

10. The dual expression vector according to claim 1, wherein said first operon comprises a first operator gene and a promoter for promoting said first operator gene, said second operon comprises a second operator gene and a promoter for promoting said second operator gene.

11. The dual expression vector according to claim 10, wherein said first operator gene is selected from LEU2, TRP1, or HIS, said second operator gene is selected from LEU2, TRP1, or HIS.

12. The dual expression vector according to claim 10, wherein said second sequence also comprises an third initiator located between said second promoter and said second gene operator, and a resistance gene connected to the transcriptional start end of said second initiator.

13. A dual expression vector comprising:
a first sequence, said first sequence comprises the orderly linked sequence consisting of a first multiple cloning site comprising SEQ ID NO: 10, a T7 promoter comprising SEQ ID NO: 9, a coding region of the GAL4 activation domain comprising SEQ ID NO: 8, a first promoter comprising SEQ ID NO: 7, a first operon comprising SEQ ID NO: 6, and a first initiator comprising SEQ ID NO: 5, said first promoter, said coding region of the GAL4 activation domain, and said first multiple cloning site have the same transcribed direction, said first promoter promotes the expression of the coding region of the GAL4 activation domain and the target genes to be inserted at said first multiple cloning site;
a second sequence, said second sequence comprises the orderly linked sequence consisting of a second multiple cloning site comprising SEQ ID NO: 12, the T7 promoter comprising SEQ ID NO: 9, a coding region of the GAL4 binding domain comprising SEQ ID NO: 13, a second promoter comprising SEQ ID NO: 14, a third initiator comprising SEQ ID NO: 1, a second operon comprising SEQ ID NO: 2, a second initiator comprising SEQ ID NO: 3 and a kanamycin resistance gene comprising SEQ ID NO: 4, said second promoter, said coding region of the GALA activation domain, and said second multiple cloning site have the same transcribed direction, said second promoter promotes the expression of the coding region of the GAL4 binding domain and the target genes to be inserted at said second multiple cloning site;
said first sequence and said second sequence are joined to form a circular DNA molecule, and said first sequence has the opposite transcribed direction from said second sequence.

14. The dual expression vector according to claim 10, wherein said vector also comprises a sequence as SEQ ID NO:15 between said kanamycin resistance gene and said first initiator, and a sequence as SEQ ID NO:16 between said first operon and said first promoter.

15. A method for testing protein-protein interactions comprising:
providing a dual expression vector;
separately amplifying the genes of different proteins;
respectively inserting the genes of said different proteins into said dual expression vector to obtain a recombinant vector;
transforming said recombinant vector into yeast to obtain a recombinant yeast; and
testing the interaction between said two different proteins on the growth state of said recombinant yeast on a selective culture medium;
wherein said dual expression vector comprising:
a first sequence, said first sequence comprises the orderly linked sequence consisting of a first multiple cloning site, a coding region of the GAL4 activation domain, a first promoter, a first operon, and a first initiator, said first promoter, said coding region of the GALA activation domain, and said first multiple cloning site have the same transcribed direction, said first promoter promotes the expression of the coding region of the GAL4 activation domain and the target genes to be inserted at said first multiple cloning site;
a second sequence, said second sequence comprises the orderly linked sequence consisting of a second multiple cloning site, a coding region of the GAL4 binding domain, a second promoter, a second operon, and a second initiator, said second promoter, said coding region of the GAL4 binding domain, and said second multiple cloning site have the same transcribed direction, said second promoter promotes the expression of the coding region of the GAL4 binding domain and the target genes to be inserted at said second multiple cloning site;
said first sequence and said second sequence are joined to form a circular DNA molecule.

16. The method according to claim 15, wherein said the target genes of said two different proteins are inserted into said first multiple cloning site, and said second multiple cloning site.

17. The method according to claim 15, wherein said yeast is Y2H yeast.

18. The method according to claim 15, wherein said selective medium is selected from SD/-Trp/-Leu, SD/-His/-Leu/-Trp, SD/-Trp/-Leu/-Ade/-His, and SD/-Trp/-Leu/-Ade/-His+3-AT.


SEQUENCE LISTING (52 sequences)

SEQ ID NO: 1 - DNA, 548 bp, synthetic construct
  source 1..548: mol_type = other DNA
atcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcat

SEQ ID NO: 2 - DNA, 1147 bp, synthetic construct
  source 1..1147: mol_type = other DNA
cctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgttctattaaatgcttcctatattatatatatagtaatgtcgttgatctatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcg

SEQ ID NO: 3 - DNA, 1346 bp, synthetic construct
  source 1..1346: mol_type = other DNA
gtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgt

SEQ ID NO: 4 - DNA, 923 bp, synthetic construct
  source 1..923: mol_type = other DNA
ggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctg

SEQ ID NO: 5 - DNA, 799 bp, synthetic construct
  source 1..799: mol_type = other DNA
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcc

SEQ ID NO: 6 - DNA, 1498 bp, synthetic construct
  source 1..1498: mol_type = other DNA
agcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtaggaaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactacctgtaccccatttaggaccacccacagcacctaacaaaacggcatcagccttcttggaggcttccagcgcctcatctggaagtgggacacctgtagcttcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcgctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagtt

SEQ ID NO: 7 - RNA, 1472 nt, synthetic construct
  source 1..1472: mol_type = other RNA
cctgcaggtcgagatccgggatcgaagaaatgatggtaaatgaaataggaaatcaaggagcatgaaggcaaaagacaaatataagggtcgaacgaaaaataaagtgaaaagtgttgatatgatgtatttggctttgcggcgccgaaaaaacgagtttacgcaattgcacaatcatgctgactctgtggcggacccgcgctcttgccggcccggcgataacgctgggcgtgaggctgtgcccggcggagttttttgcgcctgcattttccaaggtttaccctgcgctaaggggcgagattggagaagcaataagaatgccggttggggttgcgatgatgacgaccacgacaactggtgtcattatttaagttgccgaaagaacctgagtgcatttgcaacatgagtatactagaagaatgagccaagacttgcgagacgcgagtttgccggtggtgcgaacaatagagcgaccatgaccttgaaggtgagacgcgcataaccgctagagtactttgaagaggaaacagcaatagggttgctaccagtataaatagacaggtacatacaacactggaaatggttgtctgtttgagtacgctttcaattcatttgggtgtgcactttattatgttacaatatggaagggaactttacacttctcctatgcacatatattaattaaagtccaatgctagtagagaaggggggtaacacccctccgcgctcttttccgatttttttctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactcca

SEQ ID NO: 8 - DNA, 342 bp, synthetic construct
  source 1..342: mol_type = other DNA
gccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagag

SEQ ID NO: 9 - DNA, 23 bp, synthetic construct
  source 1..23: mol_type = other DNA
taatacgactcactatagggcga

SEQ ID NO: 10 - DNA, 27 bp, synthetic construct
  source 1..27: mol_type = other DNA
catatggccatggaggccagtggatcc

SEQ ID NO: 11 - DNA, 145 bp, synthetic construct
  source 1..145: mol_type = other DNA
aatgatacggcgaccaccgatatataaactcatttacttatgtaggaataaagagtatcatctttcaaagttgaaacgcgtcgctattttttagatgatactctttatttctagacagttatatacgtatgccgtcttctgcttg

SEQ ID NO: 12 - DNA, 28 bp, synthetic construct
  source 1..28: mol_type = other DNA
gagctcgagctgcagatgaatcgatacc

SEQ ID NO: 13 - DNA, 441 bp, synthetic construct
  source 1..441: mol_type = other DNA
cgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcat

SEQ ID NO: 14 - DNA, 707 bp, synthetic construct
  source 1..707: mol_type = other DNA
ggagttgattgtatgcttggtatagcttgaaatattgtgcagaaaaagaaacaaggaagaaagggaacgagaacaatgacgaggaaacaaaagattaataattgcaggtctatttatacttgatagcaagacagcaaacttttttttatttcaaattcaagtaactggaaggaaggccgtataccgttgctcattagagagtagtgtgcgtgaatgaaggaaggaaaaagtttcgtgtgcttcgagatacccctcatcagctctggaacaacgacatctgttggtgctgtctttgtcgttaattttttcctttagtgtcttccatcatttttttgtcattgcggatatggtgagacaacaacgggggagagagaaaagaaaaaaaaagaaaagaagttgcatgcgcctattattacttcaatagatggcaaatggaaaaagggtagtgaaacttcgatatgatgatggctatcaagtctagggctacagtattagttcgttatgtaccaccatcaatgaggcagtgtaattggtgtagtcttgtttagcccattatgtcttgtctggtatctgttctattgtatatctcccctccgccacctacatgttagggagaccaacgaaggtattataggaatcccgatgtatgggtttggttgccagaaaagaggaagtccatattgtacacccggaaacaacaaaaggat

SEQ ID NO: 15 - DNA, 585 bp, synthetic construct
  source 1..585: mol_type = other DNA
agcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 16 - DNA, 281 bp, synthetic construct
  source 1..281: mol_type = other DNA
gggggatctcgactctagctagaggatcaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgataacttcgtataatgtatgctatacgaagttattaggtctgaagaggagtttacgtccagccaagctagc

SEQ ID NO: 17 - DNA, 22 bp, synthetic construct
  source 1..22: mol_type = other DNA
cctgcaggtcgagatccgggat

SEQ ID NO: 18 - DNA, 22 bp, synthetic construct
  source 1..22: mol_type = other DNA
cctgcaggtcgagatccgggat

SEQ ID NO: 19 - DNA, 2016 bp, synthetic construct
  source 1..2016: mol_type = other DNA
cctgcaggtcgagatccgggatcgaagaaatgatggtaaatgaaataggaaatcaaggagcatgaaggcaaaagacaaatataagggtcgaacgaaaaataaagtgaaaagtgttgatatgatgtatttggctttgcggcgccgaaaaaacgagtttacgcaattgcacaatcatgctgactctgtggcggacccgcgctcttgccggcccggcgataacgctgggcgtgaggctgtgcccggcggagttttttgcgcctgcattttccaaggtttaccctgcgctaaggggcgagattggagaagcaataagaatgccggttggggttgcgatgatgacgaccacgacaactggtgtcattatttaagttgccgaaagaacctgagtgcatttgcaacatgagtatactagaagaatgagccaagacttgcgagacgcgagtttgccggtggtgcgaacaatagagcgaccatgaccttgaaggtgagacgcgcataaccgctagagtactttgaagaggaaacagcaatagggttgctaccagtataaatagacaggtacatacaacactggaaatggttgtctgtttgagtacgctttcaattcatttgggtgtgcactttattatgttacaatatggaagggaactttacacttctcctatgcacatatattaattaaagtccaatgctagtagagaaggggggtaacacccctccgcgctcttttccgatttttttctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactccaagctttgcaaagatggataaagcggaattaattcccgagcctccaaaaaagaagagaaaggtcgaattgggtaccgccgccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagatctttaatacgactcactatagggcgagcgccgccatggagtacccatacgacgtaccagattacgctcatatggccatggaggccagtgaattccacccgggtgggcatcgatacgggatcc

SEQ ID NO: 20 - DNA, 35 bp, synthetic construct
  source 1..35: mol_type = other DNA
gaccatgattacgccttaagcaaggattttcttaa

SEQ ID NO: 21 - DNA, 30 bp, synthetic construct
  source 1..30: mol_type = other DNA
atctcgacctgcagggctagcttggctgga

SEQ ID NO: 22 - DNA, 1782 bp, synthetic construct
  source 1..1782: mol_type = other DNA
ttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtaggaaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactacctgtaccccatttaggaccacccacagcacctaacaaaacggcatcagccttcttggaggcttccagcgcctcatctggaagtgggacacctgtagcttcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcgctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttgggggatctcgactctagctagaggatcaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgataacttcgtataatgtatgctatacgaagttattaggtctgaagaggagtttacgtccagccaagctagc

SEQ ID NO: 23 - DNA, 20 bp, synthetic construct
  source 1..20: mol_type = other DNA
ggcgtaatcatggtcatagc

SEQ ID NO: 24 - DNA, 30 bp, synthetic construct
  source 1..30: mol_type = other DNA
cgcagatataggtgcgacgtgaacagtgag

SEQ ID NO: 25 - DNA, 3620 bp, synthetic construct
  source 1..3620: mol_type = other DNA
gacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatgacgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 26 - DNA, 21 bp, synthetic construct
  source 1..21: mol_type = other DNA
gcaacacgcagatataggtgc

SEQ ID NO: 27 - DNA, 21 bp, synthetic construct
  source 1..21: mol_type = other DNA
ctgatctcagaggaggacctg

SEQ ID NO: 28 - DNA, 3354 bp, synthetic construct
  source 1..3354: mol_type = other DNA
caggtcctcctctgagatcagcttctgctcctccatgatggcggctcgccctatagtgagtcgtattacaaattccggcgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcatctttcaggaggcttgcttcaagcttggagttgattgtatgcttggtatagcttgaaatattgtgcagaaaaagaaacaaggaagaaagggaacgagaacaatgacgaggaaacaaaagattaataattgcaggtctatttatacttgatagcaagacagcaaacttttttttatttcaaattcaagtaactggaaggaaggccgtataccgttgctcattagagagtagtgtgcgtgaatgaaggaaggaaaaagtttcgtgtgcttcgagatacccctcatcagctctggaacaacgacatctgttggtgctgtctttgtcgttaattttttcctttagtgtcttccatcatttttttgtcattgcggatatggtgagacaacaacgggggagagagaaaagaaaaaaaaagaaaagaagttgcatgcgcctattattacttcaatagatggcaaatggaaaaagggtagtgaaacttcgatatgatgatggctatcaagtctagggctacagtattagttcgttatgtaccaccatcaatgaggcagtgtaattggtgtagtcttgtttagcccattatgtcttgtctggtatctgttctattgtatatctcccctccgccacctacatgttagggagaccaacgaaggtattataggaatcccgatgtatgggtttggttgccagaaaagaggaagtccatattgtacacccggaaacaacaaaaggatctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatagatcggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgttctattaaatgcttcctatattatatatatagtaatgtcgttgatctatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgc

SEQ ID NO: 29 - DNA, 40 bp, synthetic construct
  source 1..40: mol_type = other DNA
tgatctcagaggaggacctgctgcatatgcctgtcaccga

SEQ ID NO: 30 - DNA, 40 bp, synthetic construct
  source 1..40: mol_type = other DNA
cgtatgccgtcttctgcttgcagcaggggatgcagaggca

SEQ ID NO: 31 - DNA, 40 bp, synthetic construct
  source 1..40: mol_type = other DNA
acgacgtaccagattacgctagcgccgccatggagtaccc

SEQ ID NO: 32 - DNA, 40 bp, synthetic construct
  source 1..40: mol_type = other DNA
tcggtggtcgccgtatcatttgtttcaggttcagggggag

SEQ ID NO: 33 - DNA, 40 bp, synthetic construct
  source 1..40: mol_type = other DNA
tgatctcagaggaggacctgatgagttgtttttctcacct

SEQ ID NO: 34 - DNA, 43 bp, synthetic construct
  source 1..43: mol_type = other DNA
cgtatgccgtcttctgcttgctagtcgcgctccgatatctcgg

SEQ ID NO: 35 - DNA, 41 bp, synthetic construct
  source 1..41: mol_type = other DNA
tcggtggtcgccgtatcatttcactggctgagcggcacaca

SEQ ID NO: 36 - DNA, 40 bp, synthetic construct
  source 1..40: mol_type = other DNA
acgacgtaccagattacgctatggaacgacaggggatgga

SEQ ID NO: 37 - RNA, 40 nt, synthetic construct
  source 1..40: mol_type = other RNA
tgatctcagaggaggacctgatgctgcggatggaggtgca

SEQ ID NO: 38 - DNA, 43 bp, synthetic construct
  source 1..43: mol_type = other DNA
cgtatgccgtcttctgcttgctagagccagagcgcgccggcct

SEQ ID NO: 39 - DNA, 40 bp, synthetic construct
  source 1..40: mol_type = other DNA
tcggtggtcgccgtatcatttcaatccgtcggccgcgtca

SEQ ID NO: 40 - DNA, 40 bp, synthetic construct
  source 1..40: mol_type = other DNA
acgacgtaccagattacgctatgcatcgtcagccatctcc

SEQ ID NO: 41 - DNA, 40 bp, synthetic construct
  source 1..40: mol_type = other DNA
tgatctcagaggaggacctgatgcagcagcaacacctgat

SEQ ID NO: 42 - DNA, 43 bp, synthetic construct
  source 1..43: mol_type = other DNA
cgtatgccgtcttctgcttgctaactgccttcttcctcggtgc

SEQ ID NO: 43 - DNA, 41 bp, synthetic construct
  source 1..41: mol_type = other DNA
tcggtggtcgccgtatcatttcaccttcggtccgtggccag

SEQ ID NO: 44 - DNA, 40 bp, synthetic construct
  source 1..40: mol_type = other DNA
acgacgtaccagattacgctatggcgatgccgtatgcctc

SEQ ID NO: 45 - DNA, 39 bp, synthetic construct
  source 1..39: mol_type = other DNA
gatctcagaggaggacctgatggaagggtgccaactgct

SEQ ID NO: 46 - DNA, 40 bp, synthetic construct
  source 1..40: mol_type = other DNA
cgtatgccgtcttctgcttgctactggtgcgccggtaagc

SEQ ID NO: 47 - DNA, 40 bp, synthetic construct
  source 1..40: mol_type = other DNA
tcggtggtcgccgtatcatttcatctctcagctgcctcgc

SEQ ID NO: 48 - DNA, 40 bp, synthetic construct
  source 1..40: mol_type = other DNA
acgacgtaccagattacgctatggcaagatcaccaaaagg

SEQ ID NO: 49 - DNA, 40 bp, synthetic construct
  source 1..40: mol_type = other DNA
tgatctcagaggaggacctgatgtcggtggatacggagcg

SEQ ID NO: 50 - DNA, 40 bp, synthetic construct
  source 1..40: mol_type = other DNA
cgtatgccgtcttctgcttgtcaaaacagcgacgaagtac

SEQ ID NO: 51 - DNA, 40 bp, synthetic construct
  source 1..40: mol_type = other DNA
tcggtggtcgccgtatcatttcagagttgagactccaggg

SEQ ID NO: 52 - DNA, 40 bp, synthetic construct
  source 1..40: mol_type = other DNA
acgacgtaccagattacgctatggcttggaatggccgctt